SEQUENCE LISTING (3 sequences)

SEQ ID NO: 1 - protein, 340 aa, artificial
  high solubility transposase
MGKSKEISQDLRKRIVDLHKSGSSLGAISKRLAVPRSSVQTIVRKYKHHGTTQPSYRSGRRRVLSPRDERTLVRKVQINPRTTAKDLVKMLEETGTKVSISTVKRVLYRHNLKGHSARKKPLLQNRHKKARLRFATAHGDKDRTFWRNVLWSDETKIELFGHNDHRYVWRKKGEASKPKNTIPTVKHGGGSIMLWGCFAAGGTGALHKIDGSMDAVQYVDILKQHLKTSVRKLKLGRKWVFQHDNDPKHTSKVVAKWLKDNKVKVLEWPSQSPDLNPIENLWAELKKRVRARRPTNLTQLHQLCQEEWAKIHPNYCGKLVEGYPKRLTQVKQFKGNATKY

SEQ ID NO: 2 - protein, 340 aa, artificial
  SB100X
MGKSKEISQDLRKRIVDLHKSGSSLGAISKRLAVPRSSVQTIVRKYKHHGTTQPSYRSGRRRVLSPRDERTLVRKVQINPRTTAKDLVKMLEETGTKVSISTVKRVLYRHNLKGHSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMDAVQYVDILKQHLKTSVRKLKLGRKWVFQHDNDPKHTSKVVAKWLKDNKVKVLEWPSQSPDLNPIENLWAELKKRVRARRPTNLTQLHQLCQEEWAKIHPNYCGKLVEGYPKRLTQVKQFKGNATKY

SEQ ID NO: 3 - protein, 343 aa, artificial
  hsSB for recombinant expression
GPMMGKSKEISQDLRKRIVDLHKSGSSLGAISKRLAVPRSSVQTIVRKYKHHGTTQPSYRSGRRRVLSPRDERTLVRKVQINPRTTAKDLVKMLEETGTKVSISTVKRVLYRHNLKGHSARKKPLLQNRHKKARLRFATAHGDKDRTFWRNVLWSDETKIELFGHNDHRYVWRKKGEASKPKNTIPTVKHGGGSIMLWGCFAAGGTGALHKIDGSMDAVQYVDILKQHLKTSVRKLKLGRKWVFQHDNDPKHTSKVVAKWLKDNKVKVLEWPSQSPDLNPIENLWAELKKRVRARRPTNLTQLHQLCQEEWAKIHPNYCGKLVEGYPKRLTQVKQFKGNATKY

(19) United States
(12) Patent Application Publication
(10) Pub. No.: US 2022/0145332 A1
(43) Pub. Date: May 12, 2022

(54) CELL PENETRATING TRANSPOSASE

(71) Applicants: European Molecular Biology Laboratory, Heidelberg (DE); Julius-Maximilians-Universität Würzburg, Würzburg (DE)

(72) Inventors: Michael HUDECEK, Höchberg (DE); Andreas MADES, Wiesbaden (DE); Orsolya BARABAS, Gaiberg (DE); Cecilia Ines ZULIANI, Heidelberg (DE); Irma QUERQUES, Zürich (CH)

(73) Assignees: European Molecular Biology Laboratory, Heidelberg (DE); Julius-Maximilians-Universität Würzburg, Würzburg (DE)

(21) Appl. No.: 17/431,903

(22) PCT Filed: Feb. 19, 2020
(86) PCT No.: PCT/EP2020/054371

§ 371 Date: Aug. 18, 2021

(51) Int. Cl. C12N 15/90; C12N 9/22

(52) U.S. Cl. CPC C12N 15/907; C12N 2800/90; C12N 9/22

(57) ABSTRACT

The Sleeping Beauty (SB) transposon is an efficient non-viral tool for inserting transgenes into cells. Its broad utilization in gene therapy has been hampered by uncontrolled transposase gene activity and the inability to use transposase protein directly. The present invention concerns the finding that SB transposase spontaneously penetrates mammalian cells and can be delivered with transposon DNA to gene-modify various cell lines, embryonic, hematopoietic and induced pluripotent stem cells. The invention provides methods and compounds to apply the cell penetrating function of transposase in methods of genetically engineering cells as well as using the transposase as a shuttle for delivering cargo into a target cell or even into a target cell organelle. Genomic integration frequency can be titrated using the technology of the invention, which adds an additional layer of safety, opening opportunities for advanced applications in genetic engineering and gene therapy.

### FIELD OF THE INVENTION

The Sleeping Beauty (SB) transposon is an efficient non-viral tool for inserting transgenes into cells. Its broad utilization in gene therapy has been hampered by uncontrolled transposase gene activity and the inability to use transposase protein directly. The present invention concerns the finding that SB transposase spontaneously penetrates mammalian cells and can be delivered with transposon DNA to gene-modify various cell lines, embryonic, hematopoietic and induced pluripotent stem cells. The invention provides methods and compounds to apply the cell penetrating function of transposase in methods of genetically engineering cells as well as using the transposase as a shuttle for delivering cargo into a target cell or even into a target cell organelle. Genomic integration frequency can be titrated using the technology of the invention, which adds an additional layer of safety, opening opportunities for advanced applications in genetic engineering and gene therapy.

### DESCRIPTION

Genetic engineering has become a crucial technology in research, biotechnology and therapy. For efficient insertion of a genetic cargo, viral vectors are widely used. However, viral gene delivery is cumbersome, costly, and carries a risk for inflammatory responses against vector-encoded epitopes (1) and for adverse genomic changes due to preferential integration in transcribed regions (2). Non-viral genome editing nucleases (such as zinc-finger nucleases, TALENs or CRISPR/Cas9) enable programmed knock-outs and small edits by triggering DNA repair-mediated changes in the target cell genome. However, their dependence on host repair compromises their utility for insertion of large transgenes, especially in medically relevant primary cells. The mutagenic potential of inherent DNA breaks has also recently shown to create a risk for genomic rearrangements (3) and malignant transformation (4, 5).

Transposons provide a non-viral alternative for efficient gene delivery and their use in research and clinical trials is rapidly increasing. They elicit comparable transgenesis rates to retroviral and lentiviral vectors, but with reduced immunogenicity, unrestricted cargo size and unbiased genomic distribution (6-8), and they have favourable attributes regarding complexity and cost for clinical implementation.

The application of transposons for genetic engineering in vertebrates was first realized with the reconstruction of an active transposon from inactive copies in fish genomes, termed Sleeping Beauty (SB) (9). Conventionally, the SB system comprises two components that are provided as plasmid DNA vectors: one coding for the transposase and one containing the genetic cargo flanked by transposon end DNA sequences. To achieve gene transfer, both vectors must be transfected, and the transposase gene must be expressed in the target cells. After expression, the SB transposase protein specifically binds the transposon ends of the cargo vector, excises the transgene and integrates it at any TA dinucleotide site in the genome of the target cell (transposition) (FIG. 1A). In contrast to genome editing nucleases, SB inserts its genetic cargo through a direct transesterification reaction, without relying on double-strand DNA breaks and the host cell's DNA repair mechanism. Due to its high insertion efficiency in vertebrates (10), SB is a valuable tool for cancer gene discovery, transgenesis and gene therapy applications (recently reviewed elsewhere (7, 11-13)). Indeed, SB is the most advanced virus-free gene delivery tool that is already being used in clinical phase I/II trials for ex vivo engineering of therapeutic cells (6, 7, 11, 13).

The majority of these trials aim to reprogram T cells by incorporating genetic information for a chimeric antigen receptor (CAR). CARs are artificial receptors that provide T cells with new specificities against malignancy-associated antigens, and CAR T cells have shown unprecedented response rates for the treatment of leukemia and lymphoma (14, 15). The first two completed clinical trials using SB for CAR gene insertion have already provided clinical proof-of-concept (16, 17). Compared to the approved CAR T cell products, which rely on virus-based gene transfer, the use of SB resulted in comparable efficacy, with the added benefit of reduced manufacturing complexity and cost, which is crucial to increasing the availability of the technology.

However, current SB systems have an important shortcoming—the use of transposase-coding DNA causes extended protein expression (17) and can even lead to transposase gene acquisition in the target cells. This lack of control over timing and kinetics of SB transposase exposure bears the risk for ongoing and uncontrolled transposition (18-20), which raises safety concerns regarding adverse transformation of the therapeutic cell product. To ensure transposase clearance and avoid the infusion of aberrant or unstable cell products, the engineered T cells of ongoing trials are cultured for 2-4 weeks after CAR gene delivery, which reduces cell fitness and therapeutic efficacy (16, 17). Thus, there is a pressing need to improve control and safety of SB, which are also critical requirements for cell and gene therapy in general.

Previous attempts to control transposase exposure have focused on mRNA-based approaches, which shortened the time of protein expression (18, 21, 22) and reduced cellular toxicity in hematopoietic stem and progenitor cells (HSPCs) (23). However, in order to achieve maximal control of activity, the direct use of protein is desired, but this has been prohibited by challenges in recombinant protein production (24). In fact, direct delivery of genome editing nucleases has been demonstrated to improve their accuracy and control (25, 26). On the other hand, transposases are generally difficult to produce recombinantly and feature low solubility in physiological conditions, preventing efficient protein delivery. Recent reports described transfection of Mos1, Mboumar-9 and Mu transposase-DNA complexes (27-29); however, low efficiency of these enzymes in mammalian cells limits their therapeutic use. In a medically relevant setting, delivery of a piggyBac transposase fused to a viral capsid was achieved and showed moderate efficiency, with the drawback of retaining viral delivery components (30, 31). For SB, protein aggregation, low stability and solubility have remained a major bottleneck for protein production and delivery to date (24).

The patent application PCT/EP2018/072320 concerns the development of an improved SB transposase with increased solubility (hsSB). Disclosed in the document are the improved characteristics of the hsSB compared to other SB transposases and its use as a tool for gene delivery, for example, in the context of therapeutic approaches.

The aim of the present invention was therefore to improve genetic engineering approaches that are based on transposable elements, and in particular SB constructs.

### BRIEF DESCRIPTION OF THE INVENTION

Generally, and by way of brief description, the main aspects of the present invention can be described as follows:

In a first aspect, the invention pertains to a method for genetically engineering a target biological cell, the method comprising in any sequence the steps of: (i) introducing a transposon construct into the biological cell and/or providing a biological cell comprising a transposon construct; and (ii) contacting the target biological cell with a transposase protein in absence of, or without using, a protein transfection procedure or protein transfection reagent.

In a second aspect, the invention pertains to a method for the delivery of a cargo-compound into a biological cell, the method comprising, covalently or non-covalently and directly or indirectly, attaching a cargo-compound to a shuttle protein to obtain a cargo-shuttle complex, and contacting the biological cell with the cargo-shuttle complex; characterized in that the shuttle protein comprises a transposase protein sequence.

In a third aspect, the invention pertains to a use of a transposase protein in the delivery of a cargo-compound into a biological cell, wherein the transposase protein is used as a cellular shuttle protein and is covalently or non-covalently and directly or indirectly attached to the cargo-compound.

In a fourth aspect, the invention pertains to a cellular-shuttle, comprising a transposase protein covalently or non-covalently coupled to a cargo compound; or a transposase protein covalently or non-covalently coupled to a linker compound, and wherein the linker compound is suitable for the covalent or non-covalent coupling of the cellular-shuttle to a cargo compound; or a transposase protein covalently or non-covalently coupled to a linker compound, and wherein the linker compound is further covalently or non-covalently coupled to a cargo-compound.

In a fifth aspect, the invention pertains to a kit for use in the delivery of cargo-compounds into a cell, the kit comprising a shuttle protein as defined in context of the method of the second aspect of the invention or in context of the shuttle according to the fourth aspect.

In a sixth aspect, the invention pertains to a method for introducing a transposase protein into a biological cell, the method comprising contacting the cell with the transposase protein in absence of a protein transfection agent or without using a protein transfection procedure, such as electroporation.

### DETAILED DESCRIPTION OF THE INVENTION

In the following, the elements of the invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine two or more of the explicitly described embodiments or which combine the one or more of the explicitly described embodiments with any number of the disclosed and/or preferred elements. Furthermore, any permutations and combinations of all described elements in this application should be considered disclosed by the description of the present application unless the context indicates otherwise.

In a first aspect, the invention pertains to a method for genetically engineering a target biological cell, the method comprising in any sequence the steps of: (i) introducing a transposon construct into the biological cell and/or providing a biological cell comprising a transposon construct; and (ii) contacting the target biological cell with a transposase protein in absence of, or without using, a protein transfection procedure or protein transfection reagent.

According to the first aspect, the target cell is genetically engineered by performing a transposition reaction with a target genome. Such a transposition reaction automatically occurs in the presence of a transposable element (transposon construct or unit) and a transposase protein which catalyses the transposition reaction.

The invention pertains foremost to the finding that a transposase, preferably hsSB, can automatically cross a cell membrane and enter a cell nucleus and thereby mediate genome modification by transposition. Such an activity is unusual for a macro-molecule such as a transposase protein, because in prior art methods transposases required an active transfection into cells using for example protein transfection reagents or procedures such as electroporation. In context of the invention there is now provided a method for genetically engineering cells wherein the method does not comprise a step of protein transfection, in particular, it is preferred that the method does not comprise the use of a protein transfection reagent or procedure in order to introduce a transposase protein into the cell. In other words, the inventive methods comprise a step of introducing a transposase protein without using any vehicle, reagent or method that alters the penetration of proteins across a cell membrane. However, if the method includes, for unrelated reasons, a step of introducing another protein which is not a transposase required for the genetic engineering into the cell, and such introducing of such another protein is done by using protein transfection, such steps shall not be in disagreement with the invention which concerns the transfection (delivery) of transposase proteins. Hence, such additional steps may be comprised if they are for the purpose of introducing other proteins than the transposase protein required for genetic engineering. Also the method of the invention is preferred where no transposase protein is indirectly introduced into the cell via introducing a genetic expression construct encoding a transposase protein, and expressing said construct within the target cell.

The term “protein transfection” in context of the invention shall be understood to pertain broadly to any methods or reagents sufficient to introduce into a target cell a protein, which otherwise would not effectively enter said target cell. Popular protein transfection systems and reagents include commercial protein transfection reagents, such as PULSin™, ProteoJuice™, Xfect™, and BioPorter®, Pierce™ Protein Transfection Reagent (ThermoFisher), TransPass™, and methods such as electroporation of proteins.

Hence, it is preferred that in context of the invention for a genetic engineering method the transposase protein is provided (introduced into a cell) by adding the transposase protein directly to a medium in which said biological cell is contained, preferably to a cell culture medium of the target biological cell. Hence, the transposase protein in accordance with the invention is directly contacted with the target cell without using any vehicle or method that alters the penetration of proteins across a cell membrane.

The term “transposase” as used herein refers to an enzyme that is a component of a functional nucleic acid-protein complex capable of transposition and which is mediating transposition. The term “transposase” also refers to integrases from retrotransposons or of retroviral origin. A “transposition reaction” as used herein refers to a reaction where a transposon inserts into a target nucleic acid. Primary components in a transposition reaction are a transposon and a transposase or an integrase enzyme. For example, the transposase system according to the invention is preferably a so called “Sleeping Beauty (SB)” transposase. In certain aspects, the transposase is an engineered enzyme with improved characteristics such as increased enzymatic function. Some specific examples of an engineered SB transposases include, without limitation, SB10, SB11 or SB100×SB transposase (see, e.g., Mates et al., Nat. Gen. 2009, incorporated herein by reference). Other transposition systems can be used, e.g., Ty1 (Devine and Boeke, 1994, and WO 95/23875), Tn7 (Craig, 1996), Tn 10 and IS 10 (Kleckner et al. 1996), Himari mariner transposase (Lampe et al., 1996), Mos1 (Tosi and Beverley, 2000), Tc1 (Vos et al., 1996), Tn5 (Park et al., 1992), P element (Kaufman and Rio, 1992) and Tn3 (Ichikawa and Ohtsubo, 1990), bacterial insertion sequences (Ohtsubo and Sekine, 1996), retroviruses (Varmus and Brown 1989), and retrotransposon of yeast (Boeke, 1989).

In preferred embodiments of the present invention the transposase is a Sleeping Beauty (SB) transposase, and preferably is SB100X (SEQ ID NO: 2) or an enzyme derived from SB100X.

Hence, the transposase polypeptide according to the invention is a polypeptide having transposase activity, wherein the at least one mutated amino acid residue is a residue that is located between amino acid 150 and 250 of the SB transposase, preferably of the SB100X transposase.

In some embodiments it is preferable that the at least one mutated amino acid residue is at least two mutated amino acid residues, or at least three, four, five or more amino acids. It is preferable that the transposase polypeptide of the invention when its sequence is aligned with the sequence of an SB transposase, preferably SB100X, is mutated in any one of amino acids 170 to 180 and/or 207 to 217. More preferably, the at least one mutated amino acid residue is selected from amino acid 176 and/or 212 of SB transposase, preferably of SB100X. Most preferably, the at least one mutated amino acid residue is mutated into a serine residue, and preferably is C176S, or C176S and I212S.

In other embodiments, the transposase polypeptide of the invention further comprises an amino acid sequence having at least 60%, 70%, 80%, 90%, 95%, 96%, 97%, 98%, 99%, most preferably 100% sequence identity to the amino acid sequence between residues 150 to 250 as shown in SEQ ID NO: 1 (hsSB). It is preferred that the transposase polypeptide includes at least a C176 mutation, preferably C176S, compared to the sequence in SEQ ID NO: 2. Even more preferably, the transposase polypeptide further includes the mutation at position 1212, preferably I212S.

In some embodiments the transposase polypeptide of the invention comprises an amino acid sequence having at least 60%, 70%, 80%, 90%, 95%, 96%, 97%, 98%, 99%, most preferably 100% sequence identity to the full length amino acid sequence as shown in SEQ ID NO: 1 or 3 (hsSB). Preferably, although the degree of sequence identity is in some embodiments below 100%, the above indicated at least one mutation shall be present in the transposase polypeptide of the invention.

In another embodiment, the invention the self-penetrating transposase protein is a fragment of the transposase. Preferably the fragment comprises the DNA binding domain of hsSB (FIG. 18). Preferably the DNA binding domain of the transposase comprises N and/or C terminal additional amino acids, such as 10, 20, 30, 40, 50, 60, 70, 80, 90, 100, 150, 200 or more.

As used herein, the terms “identical” or percent “identity”, when used anywhere herein in the context of two or more nucleic acid or protein/polypeptide sequences, refer to two or more sequences or sub-sequences that are the same or have (or have at least) a specified percentage of amino acid residues or nucleotides that are the same (i.e., at, or at least, about 60% identity, preferably at, or at least, 65%, 70%, 75%, 80%, 85%, 90%, 91%, 92%, 93% or 94%, identity, and more preferably at, or at least, about 95%, 96%, 97%, 98%, 99%, or higher identity over a specified region—preferably over their full length sequences—, when compared and aligned for maximum correspondence over the comparison window or designated region) as measured using a sequence comparison algorithms, or by manual alignment and visual inspection (see, e.g., NCBI web site). In a particular embodiment, for example when comparing the protein or nucleic acid sequence of the transposase of the invention to for example a reference (non-mutated transposase), the percentage identity can be determined by the Blast searches provided in NCBI; in particular for amino acid identity, those using BLASTP 2.2.28+ with the following parameters: Matrix: BLOSUM62; Gap Penalties: Existence: 11, Extension: 1; Neighboring words threshold: 11; Window for multiple hits: 40.

In addition, in some embodiments, the transposase polypeptide of the invention has an increased solubility compared to a reference non-mutated transposase polypeptide, preferably wherein the reference non-mutated transposase polypeptide is SB100X transposase, preferably as shown in SEQ ID NO: 2 (non-mutated SB100X).

In some aspects and embodiment the transposon protein comprises an amino acid sequence having at least 50, 60, 70, 80, 85, 90, 95, 96, 97, 98, 99, 100% sequence identity to the amino acid sequence of any given transposase protein. Such a transposase protein consists of, or consists essentially of, and amino acid sequence shown in any one of SEQ ID NO: 1 to 3, optionally with not more than 50, 20, 10, 9, 8, 7, 6, 5, 4, 3, 2, 1 amino acid substitutions, additions, insertions, deletions or inversions, compared to these sequences. Such a variant transposase protein however still retains its transposase activity and/or its cell-penetrating activity according to the invention.

In particular embodiments of the invention a transposon construct comprises a genetic sequence to be genetically introduced into a target genome. A transposon construct or unit shall in context of the herein disclosed invention pertain to the nucleic acid (or genetic) construct comprising a target sequence that is intended to be subject of transposition in operable linkage to transposon genetic elements that are necessary for a successful transposition of the unit mediated by a transposase protein. In particular preferred embodiments, the transposon construct does not comprise a nucleotide sequence encoding for a transposase protein. Hence, a transposon construct or unit of the invention contains preferably inverted terminal repeats (ITRs) or direct terminal repeats (DTRs) that flank a sequence of interest to be inserted into the genome of a target cell (target sequence to be transposed). Usually a transposon unit will be nucleic acid and may be a vector of any form suitable for transposition.

In certain embodiments of the invention the transposable element or transposon construct or unit is introduced into the target cell, for example by using known nucleic acid transfection systems. However, the method of the invention may also be performed in a target cell which already contains the transposon construct or unit and therefore, wherein by introducing into the target cell the transposase protein in accordance with the invention, the transposition reaction is initiated.

As used herein, the term “inverted terminal repeat” refers to a sequence located at one end of a transposon unit that can be cleaved by a transposase polypeptide when used in combination with a complementary sequence that is located at the opposing end of the vector or transposon unit. The pair of inverted terminal repeats is involved in the transposition activity of the transposon of the transposon unit of the present disclosure, in particular involved in DNA addition or removal and excision and integration of DNA of interest. In one example, at least one pair of an inverted terminal repeat appears to be the minimum sequence required for transposition activity. In another example, the transposon unit of the present disclosure may comprise at least two, three or four pairs of inverted terminal repeats. As would be understood by the person skilled in the art, to facilitate ease of cloning, the necessary terminal sequence may be as short as possible and thus contain as little inverted repeats as possible. Thus, in one example, the transposon unit of the present disclosure may comprise not more than one, not more than two, not more than three or not more than four pairs of inverted terminal repeats. In one example, the transposon unit of the present disclosure may comprise only one inverted terminal repeat. Whilst not wishing to be bound by theory, it is envisaged that having more than one pair of inverted terminal repeats may be disadvantageous as it may lead to non-specific transposase binding to the multiple inverted terminal repeats and resulting in the removal of desired sequence or insertion of undesirable sequences. The inverted terminal repeat of the present disclosure may form either a perfect inverted terminal repeat (or interchangeably referred to as “perfect inverted repeat”) or imperfect inverted terminal repeat (or interchangeably referred to as “imperfect inverted repeat”). As used herein, the term “perfect inverted repeat” refers to two identical DNA sequences placed at opposite direction. The above descriptions for transposon units with ITR also apply for transposon units including DTRs.

A transposon system (or unit) that could be used with the inventive systems and components of the invention is for example disclosed in WO 2017/050448 Ai, which is included in the present disclosure by reference in its entirety.

A transposon construct according to the invention is preferable, wherein said transposon unit is provided in the form of a minicircle. However, the transposon unit may be other nucleic acid systems. However, minicircles are preferable in the context of T cell engineering, for example for the introduction of CAR into a T cell.

In preferred aspects and embodiment of the invention, the target sequence to be introduced into the genome of the target cell by transposition is a sequence encoding for a CAR, an antibody or a T cell receptor. Or any variant of such molecules. Hence, in some embodiments the methods and compounds of the invention are preferably used for genetically engineering T cells to generate CAR T cells. As used herein, the term “Chimeric Antigen Receptor T cells” also called CAR T cells refers to lymphocytes which express Chimeric Antigen Receptor (CAR). Hence, the methods of the invention include introducing all necessary genetic elements for the expression of the CAR in the target cell. The term “Chimeric Antigen Receptor” or “CAR” has its general meaning in the art and refers to an artificially constructed hybrid protein or polypeptide containing the antigen binding domains of an antibody (e.g., scFv) linked to T cell signalling domains. Characteristics of CARs include their ability to redirect T cell specificity and reactivity toward a selected target in a non-MHC-restricted manner, exploiting the antigen-binding properties of monoclonal antibodies. The non-MHC-restricted antigen recognition gives T cells expressing CARs the ability to recognize antigen independently of antigen processing, thus bypassing a major mechanism of tumour escape. Moreover, when expressed in T-cells, CARs advantageously do not dimerize with endogenous T cell receptor (TCR) alpha and beta chains. Strategies to design and produce such CARs are well known in the art, references can be found for example in Bonini and Mondino, Eur. J. Immunol. 2015 (19), Srivastava and Riddell, Trends Immunol. 2015 (20), Jensen and Riddell, Curr. Opin. Immunol. 2015 (21), Gill and June, Immunol. Rev. 2015 (22).

The transposon system of the invention in preferred embodiments is an SB transposon system.

A target cell in accordance with the invention is preferably selected from a mammalian cell, preferably selected from a stem cell, such as a hematopoietic stem cell, embryonic stem cell, spontaneously immortalized cell, artificial immortalized cell, primary cell (neurons, resting T cells), a cell derived from a B-cell such as plasma cells, Chinese hamster ovary (CHO) cell, induced pluripotent stem cell (iPSC), or is an immune cell, such as a T lymphocyte, preferably a CD4 or CD8 positive T cell, or is a Natural Killer (NK) cell, a macrophage, a dendritic cell or a B-cell.

In a second aspect, the invention pertains to a method for the delivery of a cargo-compound into a biological cell, the method comprising, covalently or non-covalently and directly or indirectly, attaching a cargo-compound to a shuttle protein to obtain a cargo-shuttle complex, and contacting the biological cell with the cargo-shuttle complex; characterized in that the shuttle protein comprises a transposase protein sequence.

In the second aspect of the invention the use of the cell penetrating activity of the transposase protein is used as a cellular shuttle to transport a cargo of any kind into a target cell. By simply attaching such cargo to the transposase protein of the invention any compound can be efficiently transported into cells. Hence, the transposase protein in accordance with the invention is used as a cellular transfection vehicle.

In preferred embodiments of the invention the cargo-compound is delivered into a biological cell and into the cell nucleus of the biological cell. However, alternatively, by changing the organelle targeting sequence in the transposase, for example exchanging the nuclear localization signal with a signal peptide of a different organelle, it is possible to target the cargo-shuttle complex to a different organelle, such as the mitochondrion, endoplasmic reticulum, Golgi etc. In certain embodiments the shuttle protein therefore comprises a deletion or mutation of a nuclear localization signal, or does not comprise a nuclear localization signal, and optionally comprises a signal sequence for the intracellular delivery into an organelle other than the cell nucleus.

The transposase used in this aspect is preferably a transposase as described herein for the other aspects and embodiments.

The cellular-shuttle of the invention in particular embodiments comprises the transposase protein which is covalently or non-covalently coupled to a linker compound, preferably wherein the linker compound is suitable for covalently or non-covalently coupling the cargo-compound to the shuttle protein. A linker may be a simple peptide linker, or may contain any functionality that facilitates the conjugation of the cargo to the shuttle protein. For example, the linker compound can be selected from a sortase donor or acceptor site, a biotin or streptavidin protein, or a functionally alternative component of a protein coupling system. Many of such systems are known to the skilled artisan and shall include the introduction of a specific functionality for chemical crosslinking, such as a cysteine residue, intein or an unnatural amino acid. Alternatively, it could also be a specific peptide suitable for non-covalent attachment of a cargo-compound (i.e. specific binding domain for DNA/RNA/chemicals/lipids/etc.).

In principle, the cell penetrating activity of the transposase of the invention can be used to transport any protein across a cellular membrane. Such cargo-compound is selected from a small molecule, a macromolecule, a peptide, a polypeptide, a protein, a nucleic acid, such as an RNA, DNA, RNA-DNA hybrid, PNA, or is a sugar compound, a fatty acid containing compound.

Similar to the above described embodiments of the first aspect of the invention, also the method of the second aspect is a method that preferably does not require the addition of a protein transfection agent or procedure, preferably wherein the method does not comprise the use of a protein transfection reagent or procedure, such as electroporation.

In a third aspect, the invention pertains to a use of a transposase protein in the delivery of a cargo-compound into a biological cell, wherein the transposase protein is used as a cellular shuttle protein and is covalently or non-covalently and directly or indirectly attached to the cargo-compound.

Preferably for the delivery no protein transfection reagents or protein transfection procedures, such as electroporation, are required or comprised. In this context the above descriptions with respect to the first and second aspect of the invention, and the embodiment that no protein transfection is required for introducing a transposase protein into a cell or cell organelle is referenced here.

In a fourth aspect, the invention pertains to a cellular-shuttle, comprising a transposase protein covalently or non-covalently coupled to a cargo compound; or a transposase protein covalently or non-covalently coupled to a linker compound, and wherein the linker compound is suitable for the covalent or non-covalent coupling of the cellular-shuttle to a cargo compound; or a transposase protein covalently or non-covalently coupled to a linker compound, and wherein the linker compound is further covalently or non-covalently coupled to a cargo-compound.

In a fifth aspect, the invention pertains to a kit for use in the delivery of cargo-compounds into a cell, the kit comprising a shuttle protein as defined in context of the method of the second aspect of the invention or in context of the shuttle according to the fourth aspect.

In a sixth aspect, the invention pertains to a method for introducing a transposase protein into a biological cell, the method comprising contacting the cell with the transposase protein in absence of a protein transfection agent or without using a protein transfection procedure, such as electroporation.

In addition to the above described aspects and embodiments, the invention in addition pertains to the following set of items:

Item 1: A method for genetically engineering a target biological cell, the method comprising in any sequence the steps of: (i) introducing a transposon construct into the biological cell and/or providing a biological cell comprising a transposon construct; and (ii) contacting the target biological cell with a transposase protein in absence of, or without using, a protein transfection procedure or protein transfection reagent.
Item 2: The method according to item 1, wherein the transposon construct comprises a genetic sequence to be genetically introduced into a target genome.
Item 3: The method according to item 1 or 2, wherein the transposase protein is, or is derived from, a Sleeping Beauty (SB) transposase.
Item 4: The method according to item 3, wherein the SB transposase is SB100X, preferably according to the amino acid sequence shown in SEQ ID NO: 2.
Item 5: The method according to item 3, wherein the SB transposase is highly soluble SB100X (hsSB) which comprises at least one mutated amino acid residue compared to the amino acid sequence between amino acid 150 and 250 of a reference non-mutated SB transposase, for example wherein the reference non-mutated SB transposase comprises the sequence shown in SEQ ID NO: 2.
Item 6: The method according to item 5, wherein the at least one mutated amino acid residue is at least two mutated amino acid residues.
Item 7: The method according to item 5 or 6, wherein the at least one mutated amino acid residue is a mutation of any one of amino acids 170 to 180 and/or 207 to 217 of SB transposase, preferably of SB100X (SEQ ID NO:2).
Item 8: The method according to any one of items 5 to 7, wherein the at least one mutated amino acid residue is selected from amino acid 176 and/or 212 of SB transposase, preferably of SB100X (SEQ ID NO:2).
Item 9: The method according to any one of items 5 to 8, wherein the at least one mutated amino acid residue is mutated into a serine residue, and preferably is C176S and I212S.
Item 10: The method according to any one of items 5 to 9, wherein the transposase protein further comprises an amino acid sequence having at least 60% sequence identity to the amino acid sequence between residues 150 to 250, preferably to the full length sequence, shown in SEQ ID NO: 1 or SEQ ID NO: 3.
Item 11: The method according to any one of items 1 to 10, wherein the shuttle protein comprises an amino acid sequence having at least 50, 60, 70, 80, 85, 90, 95, 96, 97, 98, 99, 100% sequence identity to the amino acid sequence of the transposase protein.
Item 12: The method according to any one of items 1 to 11, wherein the shuttle protein consists of, or consists essentially of, an amino acid sequence shown in any one of SEQ ID NO: 1 to 3, optionally with not more than 20, 10, 9, 8, 7, 6, 5, 4, 3, 2, 1 amino acid substitutions, additions, insertions, deletions or inversions, compared to these sequences.
Item 13: The method according to any one of items 1 to 12, wherein the transposase protein is provided by adding the transposase protein to a medium in which said biological cell is contained, preferably to a cell culture medium of the target biological cell.
Item 14: The method according to any one of items 1 to 13, wherein the target biological cell is a mammalian cell, preferably selected from a stem cell, such as a hematopoietic stem cell, embryonic stem cell, spontaneously immortalized cell, artificial immortalized cell, primary cell (neurons, resting T cells), a cell derived from a B-cell such as plasma cells, Chinese hamster ovary (CHO) cell, induced pluripotent stem cell (iPSC), or is an immune cell, such as a T lymphocyte, preferably a CD4 or CD8 positive T cell, or is a Natural Killer (NK) cell, a macrophage, a dendritic cell or a B-cell.
Item 15: The method according to any one of items 1 to 14, wherein the transposon comprises a protein encoding nucleotide sequence, such as a sequence encoding for an antibody, a T cell receptor, or a chimeric antigen receptor (CAR).
Item 16: A method for the delivery of a cargo-compound into a biological cell, the method comprising, covalently or non-covalently and directly or indirectly, attaching a cargo-compound to a shuttle protein to obtain a cargo-shuttle complex, and contacting the biological cell with the cargo-shuttle complex; characterized in that the shuttle protein comprises a transposase protein sequence.
Item 17: The method according to item 16, wherein the cargo-compound is delivered into a biological cell and into the cell nucleus of the biological cell.
Item 18: The method according to item 16 or 17, wherein the transposase protein sequence is derived from a Sleeping Beauty (SB) transposase.
Item 19: The method according to item 18, wherein the SB transposase is SB100X, preferably according to the amino acid sequence shown in SEQ ID NO: 2.
Item 20: The method according to item 18, wherein the SB transposase is highly soluble SB100X (hsSB) which comprises at least one mutated amino acid compared to the amino acid sequence between amino acid 150 and 250 of a reference non-mutated SB transposase, such as the sequence shown in SEQ ID NO: 2.
Item 21: The method according to item 20, wherein the at least one mutated amino acid residue is at least two mutated amino acid residues.
Item 22: The method according to item 20 or 21, wherein the at least one mutated amino acid residue is a mutation of any one of amino acids 170 to 180 and/or 207 to 217 of SB transposase, preferably of SB100X (SEQ ID NO:2).
Item 23: The method according to any one of items 20 to 22, wherein the at least one mutated amino acid residue is selected from amino acid 176 and/or 212 of SB transposase, preferably of SB100X (SEQ ID NO:2).
Item 24: The method according to any one of items 20 to 23, wherein the at least one mutated amino acid residue is mutated into a serine residue, and preferably is C176S and I212S. Item 25: The method according to any one of items 20 to 24, wherein the shuttle protein further comprising an amino acid sequence having at least 60% sequence identity to the amino acid sequence between residues 150 to 250, preferably to the full length sequence, shown in SEQ ID NO: 1 or SEQ ID NO: 3.
Item 26: The method according to any one of items 16 to 25, wherein the shuttle protein comprises an amino acid sequence having at least 50, 60, 70, 80, 85, 90, 95, 96, 97, 98, 99, 100% sequence identity to the amino acid sequence of the transposase protein.
Item 27: The method according to any one of items 16 to 20, wherein the shuttle protein comprises an amino acid sequence having at least 50, 60, 70, 80, 85, 90, 95, 96, 97, 98, 99, 100% sequence identity with at least 50, preferably 100, 150, 200, preferably at least 300 consecutive amino acids of the transposase protein.
Item 28: The method according to any one of items 16 to 27, wherein the shuttle protein consists of, or consists essentially of, an amino acid sequence shown in any one of SEQ ID NO: 1 to 3, optionally with not more than 20, 10, 9, 8, 7, 6, 5, 4, 3, 2, 1 amino acid substitutions, additions, insertions, deletions or inversions, compared to these sequences.
Item 29: The method according to any one of items 16 to 28, wherein the shuttle protein is covalently or non-covalently coupled to a linker compound, preferably wherein the linker compound is suitable for covalently or non-covalently coupling the cargo-compound to the shuttle protein.
Item 30: The method according to item 29, wherein the linker compound is a selected from a sortase donor or acceptor site, a biotin or streptavidin protein, or a functionally alternative component of a protein coupling system.
Item 31: The method according to any one of items 16 to 30, wherein the cargo-compound is selected from a small molecule, a macromolecule, a peptide, a polypeptide, a protein, a nucleic acid, such as an RNA, DNA, RNA-DNA hybrid, PNA, or is a sugar compound, a fatty acid containing compound.
Item 32: The method according to any one of the preceding items, wherein the shuttle protein comprises a deletion or mutation of a nuclear localization signal, or does not comprise a nuclear localization signal, and optionally comprises a signal sequence for the intracellular delivery into an organelle other than the cell nucleus.
Item 33: The method according to any one of the preceding items, wherein the method does not require the addition of a protein transfection agent or procedure, preferably wherein the method does not comprise the use of a protein transfection reagent or procedure, such as electroporation.
Item 34: The method according to any one of the preceding items, wherein the biological cell is a mammalian cell.
Item 35: A use of a transposase protein in the delivery of a cargo-compound into a biological cell, wherein the transposase protein is used as a cellular shuttle protein and is covalently or non-covalently and directly or indirectly attached to the cargo-compound.
Item 36: The use according to item 35, wherein for the delivery no protein transfection reagents or protein transfection procedures, such as electroporation, are required or comprised.
Item 37: The use according to item 35 or 36, wherein the transposase protein is a shuttle protein as defined in any one of method items 16 to 34.
Item 38: A cellular-shuttle, comprising
(i) a transposase protein covalently or non-covalently coupled to a cargo compound; or
(ii) a transposase protein covalently or non-covalently coupled to a linker compound, and wherein the linker compound is suitable for the covalent or non-covalent coupling of the cellular-shuttle to a cargo compound; or
(iii) a transposase protein covalently or non-covalently coupled to a linker compound, and wherein the linker compound is further covalently or non-covalently coupled to a cargo-compound.
   Item 39: The cellular-shuttle according to item 16, wherein the transposase protein is a shuttle protein as defined in any one of items 16 to 34.
   Item 40: The cellular-shuttle according to item 38 or 39, wherein the cargo-compound is selected from a small molecule, a macro-molecule, a peptide, a polypeptide, a protein, a nucleic acid, such as an RNA, DNA, RNA-DNA hybrid, PNA, or is a sugar compound, a fatty acid containing compound.
   Item 41: A kit for use in the delivery of cargo-compounds into a cell, the kit comprising a shuttle protein as defined in any one of items 16 to 34, or a cellular-shuttle according to any one of items 38 to 40.
   Item 42: A method for introducing a transposase protein into a biological cell, the method comprising contacting the cell with the transposase protein in absence of a protein transfection agent or without using a protein transfection procedure, such as electroporation.
   Item 43: The method according to item 42, wherein the transposase protein is a transposase protein as defined in any one of items 16 to 34.
   Item 44: The method according to item 42 or 43, wherein the transposase protein is a recombinantly expressed protein and added to the cell culture medium of the biological cell.

The terms “of the [present] invention”, “in accordance with the invention”, “according to the invention” and the like, as used herein are intended to refer to all aspects and embodiments of the invention described and/or claimed herein.

As used herein, the term “comprising” is to be construed as encompassing both “including” and “consisting of”, both meanings being specifically intended, and hence individually disclosed embodiments in accordance with the present invention. Where used herein, “and/or” is to be taken as specific disclosure of each of the two specified features or components with or without the other. For example, “A and/or B” is to be taken as specific disclosure of each of (i) A, (ii) B and (iii) A and B, just as if each is set out individually herein. In the context of the present invention, the terms “about” and “approximately” denote an interval of accuracy that the person skilled in the art will understand to still ensure the technical effect of the feature in question. The term typically indicates deviation from the indicated numerical value by ±20%, ±15%, ±10%, and for example f5%. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. Where an indefinite or definite article is used when referring to a singular noun, e.g. “a”, “an” or “the”, this includes a plural of that noun unless something else is specifically stated.

It is to be understood that application of the teachings of the present invention to a specific problem or environment, and the inclusion of variations of the present invention or additional features thereto (such as further aspects and embodiments), will be within the capabilities of one having ordinary skill in the art in light of the teachings contained herein.

Unless context dictates otherwise, the descriptions and definitions of the features set out above are not limited to any particular aspect or embodiment of the invention and apply equally to all aspects and embodiments which are described.

All references, patents, and publications cited herein are hereby incorporated by reference in their entirety.

### BRIEF DESCRIPTION OF THE FIGURES AND SEQUENCES

The figures show:

FIG. 1 shows a schematic representation of genome engineering by the SB transposase. LE and RE mark the left and right transposon end sequences, respectively. Cargo gene transfer in the target genome is executed by the transposase, expressed from a plasmid vector (bent arrow) in the target cells.

FIG. 2 shows that direct hsSB delivery allows for efficient transgenesis in diverse mammalian cells and stem cells. Representative flow cytometric analysis of HeLa cells (top panel), Chinese hamster ovary (CHO) cells (middle panel) and mouse embryonic stem cells (mESCs; bottom panel) transfected with Venus-carrying transposon plasmids and electroporated with hsSB transposase. Cells stably expressing an integrated Venus gene were identified 3 weeks post-transfection. The electroporated hsSB protein amounts are indicated above. Y-axis: propidium iodide (PI) staining to exclude dead cells; x-axis: green fluorescence from Venus; NT, non-transfected.

FIG. 3 shows transgenesis efficiency of the a system containing recombinantly expressed SB protein with any transgene vector (SBprotAct) in different cell lines, quantified by flow cytometry. Errors bars indicate the standard deviation (n=2).

FIG. 4 shows a schematic representation of the cell engineering procedure of the invention, using spontaneous hsSB penetration.

FIG. 5 shows immunofluorescence imaging of hsSB-treated (top) and non-treated (bottom) HeLa cells, showing DAPI-stained nuclei (left), hsSB staining (middle) and the merge (right). Arrows mark cells with hsSB in the nucleus.

FIG. 6 shows Western blot analysis showing cellular uptake and retention of hsSB in HeLa cells upon addition to the culture media. Samples were blotted with either anti-SB antibody or anti-GAPDH (glyceraldehyde 3-phosphate dehydrogenase) as internal loading control.

FIG. 7 shows a representative flow cytometric analysis of HeLa cells transfected with Venus-encoding transposon MC and incubated with hsSB in the culture media. Venus positive cells were sorted after 2 days and analyzed 3 weeks post-delivery. Y-axis: 4′,6-diamidino-2-phenylindole (DAPI) staining to exclude dead cells; x-axis: green fluorescence from Venus. hsSB protein concentration in the culture media are indicated above each plot. NT, non-transfected.

FIG. 8 shows a Western blot analysis of induced pluripotent stem cells (iPSCs) with anti-SB antibody, following hsSB penetration from the culture media.

FIG. 9 shows a representative flow cytometric analysis of iPSCs 3 weeks after transfection with Venus transposon MC and incubation with hsSB.

FIG. 10 shows a schematic representation of T cell engineering procedure, using spontaneous hsSB penetration.

FIG. 11 shows immunofluorescence imaging of T cells showing DAPI-stained nuclei (left), hsSB staining (middle) and the merge (right). Cells stained in absence of primary SB antibody are shown below (IF control).

FIG. 12 shows a representative flow cytometric analysis of CD8+ T cells transfected with transposon minicircles (MC) and incubated with hsSB. CD8+ T cells from healthy donors were transfected with CD19 CAR MC and enriched for CAR-positive cells (using EGFRt as marker) by magnetic associated cell sorting (MACS). Representative FACS plots from one of 3 experiments (from 3 different T cell donors) are shown with fluorescence from CD8 and EGFRt specific antibodies (CD8-VioBlue and EGFRt-AF647, respectively) plotted. hsSB protein concentration in the culture media are indicated above each plot. NT, non-transfected.

FIG. 13 shows the cytolytic activity of CD19 CAR T cells generated by hsSB penetration or MC-MC controls. Cytolysis was calculated from the luminescence signals of ffLuc-expressing target cells in a 5 h co-culture assay in the presence of excess luciferin. NT, non-transfected. E:T ratio, effector to target ratio.

FIG. 14 shows the average number of CAR transgene insertions as measured by digital droplet PCR (ddPCR) of CAR T cell genomic DNA. Error bars show the copy number estimates of two independent ddPCR assays (performed on same genomic DNA samples) at 95% confidence intervals.

FIG. 15: shows penetration of hsSB-GFP fusion protein. (A) fluorescence imaging of HeLa cells showing hsSB-GFP (left) and DAPI-stained nuclei (right) following 1 h incubation with the protein. Scale bar 20 m. (B) shows fluorescence imaging of HeLa cells showing hsSB-GFP (left) and DAPI-stained nuclei (right) 24 h later. Scale bar 20 m.

FIG. 16 shows penetration of an hsSB catalytically inactive mutant fused to the N-terminus of GFP. (A) fluorescence imaging of HeLa cells showing hsSB-D153N-D244N-GFP (left) and DAPI-stained nuclei (right) following 1 h incubation with the protein. Scale bar 20 m. (B) fluorescence imaging of HeLa cells showing hsSB-D153N-D244N-GFP (left) and DAPI-stained nuclei (right) 24 h later. Scale bar 20 m.

FIG. 17 shows penetration of GFP-hsSB fusion protein. (A) fluorescence imaging of HeLa cells showing GFP-hsSB (left) and DAPI-stained nuclei (right) following 1 h incubation with the protein. Scale bar 20 μm. (B) fluorescence imaging of HeLa cells showing GFP-hsSB (left) and DAPI-stained nuclei (right) 24 h later. Scale bar 20 m.

FIG. 18 shows that the N-terminal DNA-binding domain (DBD) of hsSB efficiently penetrates into HeLa cells. (A) immunofluorescence imaging of HeLa cells showing SB staining (left) and DAPI-stained nuclei (right) following 3 h incubation with the protein. Scale bar 20 μm. A schematic of the construct hsSB-1-123 is shown below (B) immunofluorescence imaging of HeLa cells showing SB staining (left) and DAPI-stained nuclei (right) 24 h later. Scale bar 20 m.

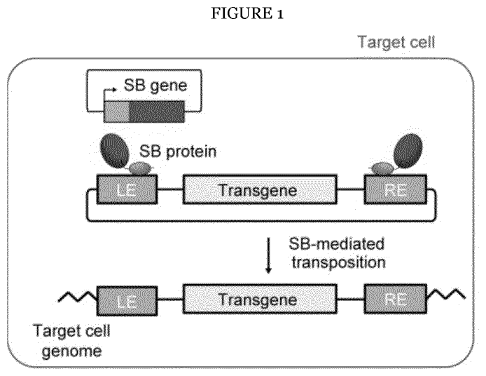

The sequences show:

SEQ ID NO: 1
shows the hsSB
MGKSKEISQDLRKRIVDLHKSGSSLGAISKRLAVPRSSVQTIVRKYKHHG
TTQPSYRSGRRRVLSPRDERTLVRKVQINPRTTAKDLVKMLEETGTKVSI
STVKRVLYRHNLKGHSARKKPLLQNRHKKARLRFATAHGDKDRTFWRNVL
WSDETKIELFGHNDHRYVWRKKGEASKPKNTIPTVKHGGGSIMLWGCFAA
GGTGALHKIDGSMDAVQYVDILKQHLKTSVRKLKLGRKWVFQHDNDPKHT
SKVVAKWLKDNKVKVLEWPSQSPDLNPIENLWAELKKRVRARRPTNLTQL
HQLCQEEWAKIHPNYCGKLVEGYPKRLTQVKQFKGNATKY
SEQ ID NO: 2
(non-mutated SB100X)
MGKSKEISQDLRKRIVDLHKSGSSLGAISKRLAVPRSSVQTIVRKYKHHG
TTQPSYRSGRRRVLSPRDERTLVRKVQINPRTTAKDLVKMLEETGTKVSI
STVKRVLYRHNLKGHSARKKPLLQNRHKKARLRFATAHGDKDRTFWRNVL
WSDETKIELFGHNDHRYVWRKKGEACKPKNTIPTVKHGGGSIMLWGCFAA
GGTGALHKIDGIMDAVQYVDILKQHLKTSVRKLKLGRKWVFQHDNDPKHT
SKVVAKWLKDNKVKVLEWPSQSPDLNPIENLWAELKKRVRARRPTNLTQL
HQLCQEEWAKIHPNYCGKLVEGYPKRLTQVKQFKGNATKY
SEQ ID NO: 3
(hsSB for recombinant expression) [symbol]
MGKSKEISQDLRKRIVDLHKSGSSLGAISKRLAVPRSSVQTIVRKYK
HHGTTQPSYRSGRRRVLSPRDERTLVRKVQINPRTTAKDLVKMLEETGTK
VSISTVKRVLYRHNLKGHSARKKPLLQNRHKKARLRFATAHGDKDRTFWR
NVLWSDETKIELFGHNDHRYVWRKKGEASKPKNTIPTVKHGGGSIMLWGC
FAAGGTGALHKIDGSMDAVQYVDILKQHLKTSVRKLKLGRKWVFQHDNDP
KHTSKVVAKWLKDNKVKVLEWPSQSPDLNPIENLWAELKKRVRARRPTNL
TQLHQLCQEEWAKIHPNYCGKLVEGYPKRLTQVKQFKGNATKY
(underlined are mutated or to-be mutated residues.
Bold and italic are residues introduced for
recombinant protein expression)

### EXAMPLES

Certain aspects and embodiments of the invention will now be illustrated by way of example and with reference to the description, figures and tables set out herein. Such examples of the methods, uses and other aspects of the present invention are representative only, and should not be taken to limit the scope of the present invention to only such representative examples.

The examples show:

### Example 1 (Comparative): Efficient Transgenesis in Mammalian Cells Using hsSB Transposase

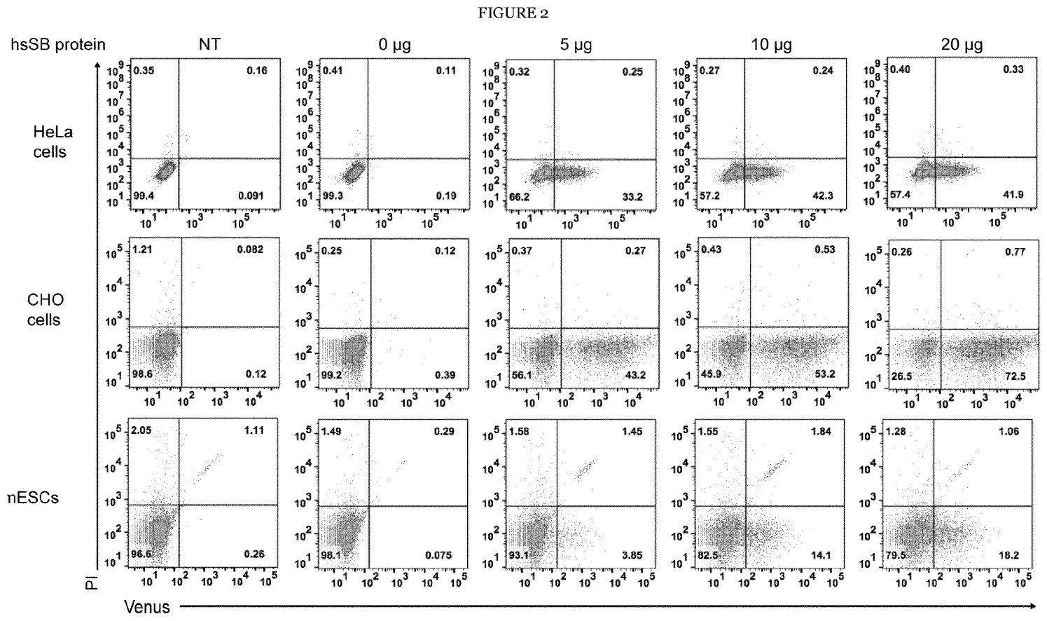

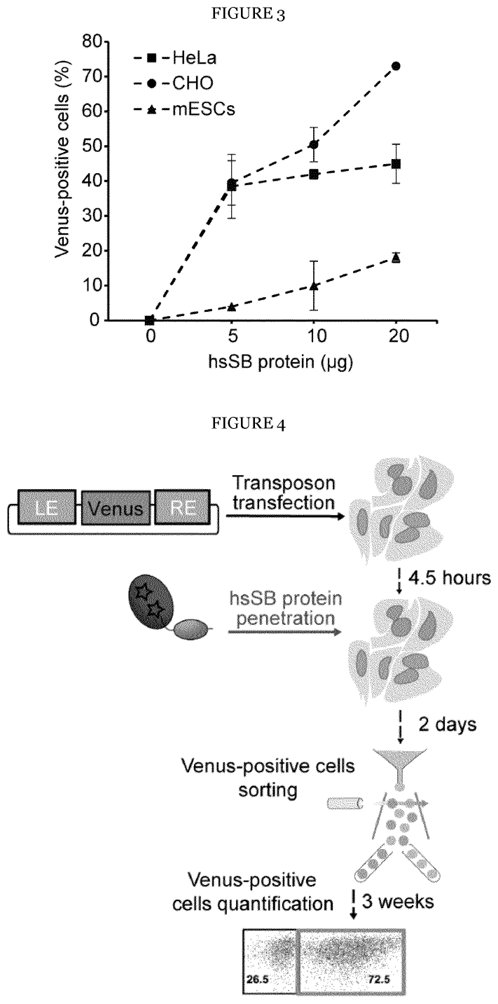

A high solubility Sleeping Beauty (hsSB) transposase developed by the inventors was tested in various mammalian cells lines for its ability of genetically engineering cells. The amino acid sequence of the improved hsSB transposase is shown in SEQ ID NO: 3. To better quantify hsSB-mediated transposition, the inventors applied a fluorescent reporter system and transfected HeLa cells with a transposon plasmid containing the Venus gene, followed by hsSB protein delivery by protein electroporation. Cells that acquired the transposon plasmid were selected by fluorescence activated cell sorting 2 days post-transfection. The transposition efficiency was then quantified three weeks later by flow cytometric analysis of green fluorescent cells that stably expressed the Venus reporter gene as a consequence of genomic insertion by hsSB (FIG. 2). A clear, dose-dependent increase in the percentage of fluorescent cells, with the maximum efficiency (42%) achieved with 20 μg of hsSB protein (FIG. 2, upper panel, and FIG. 3) was detected. Also Chinese hamster ovary (CHO) cells and mouse embryonic stem cells could be efficiently transfected with the hsSB transposase of the invention (FIGS. 2 and 3).

### Example 2: Transposase has Intrinsic Cell Penetrating Properties

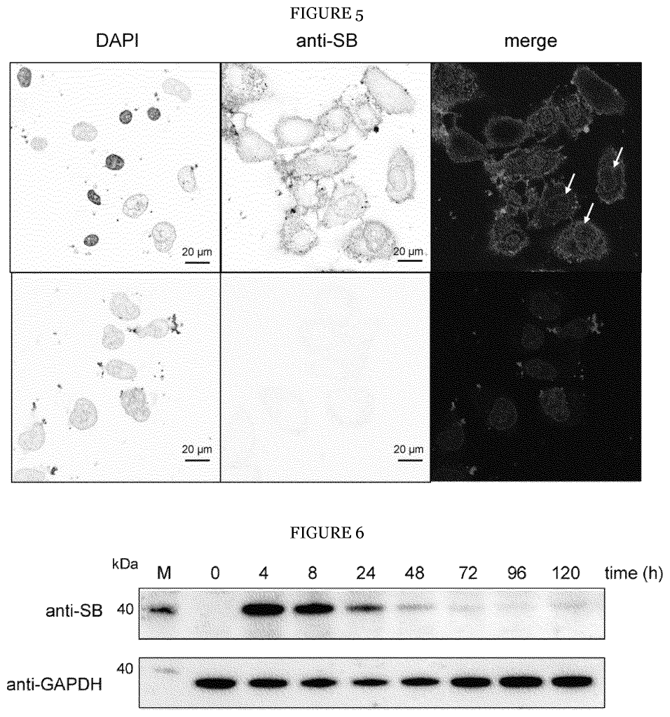

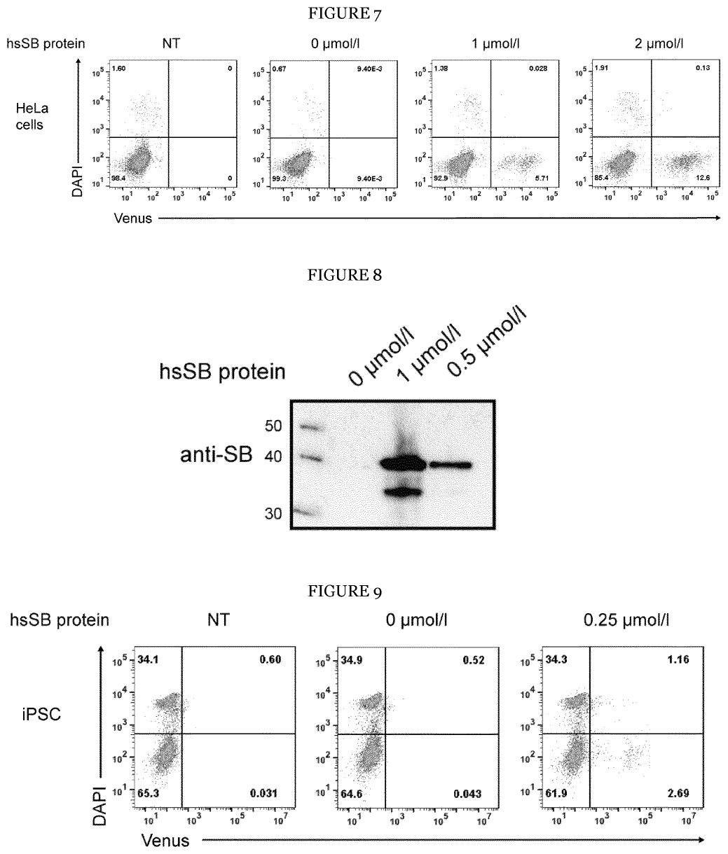

For further developing methods for the genetic engineering of mammalian cells the inventors sought to make transposase delivery simpler and gentler. Remarkably, the inventors observed that the transposase protein autonomously penetrates HeLa cells and enters the nucleus when simply added to the culture medium (FIGS. 4 and 5). To test if hsSB can mediate transposition when delivered this way, the inventors transfected HeLa cells with a MC containing the Venus gene and then added hsSB to the culture medium without a further pulse or use of a transfection reagent (FIG. 4). Longitudinal Western blot analysis showed hsSB uptake within 4 hours, followed by clearance already 24 hours after delivery (FIG. 6). Fluorescent cell sorting 3 weeks post transfection revealed up to 12% Venus-positive cells (FIG. 7), demonstrating that hsSB mediated efficient transgene integration.

Next, a similar procedure for genetic engineering of human iPSCs was tested. iPSCs offer great potential for regenerative medicine but are among the most difficult cells to engineer due to their sensitivity to transfection procedures. The inventors first transfected the iPSCs with a Venus-carrying MC using a stem cell specific transfection reagent and then incubated them with hsSB protein-containing medium to allow protein penetration in the cells. hsSB efficiently penetrated iPSCs (FIG. 8) and flow cytometry of the treated cells after three weeks revealed remarkable transgenesis efficiencies of up to 3.31% (calculated as the percentage of stable integrants at 3 weeks over all transfected cells, FIG. 9). This shows that hsSB's non-invasive cell penetration helps to modify iPSCs.

### Example 3: Novel Genetic Engineering Method can be Used to Generate CAR-T Cells

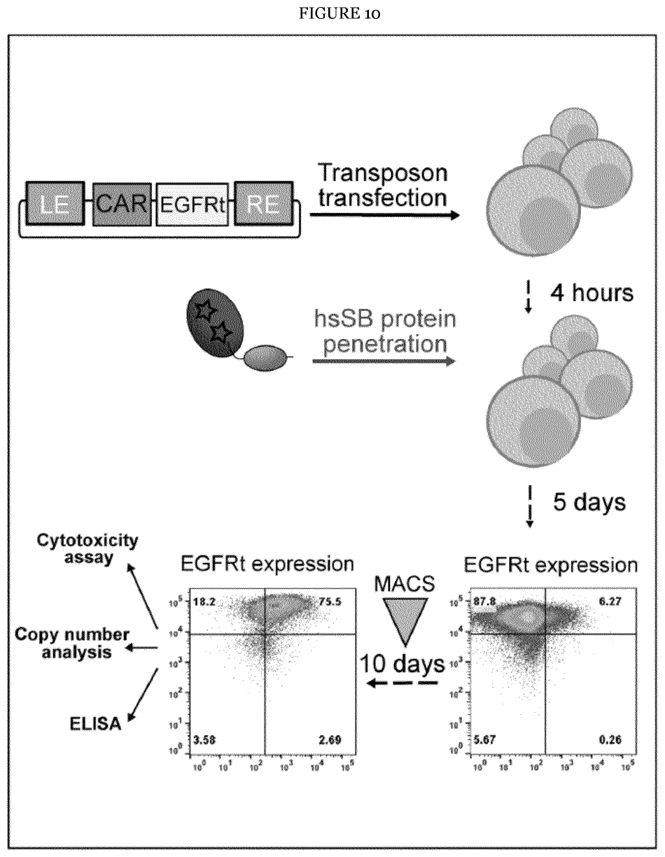

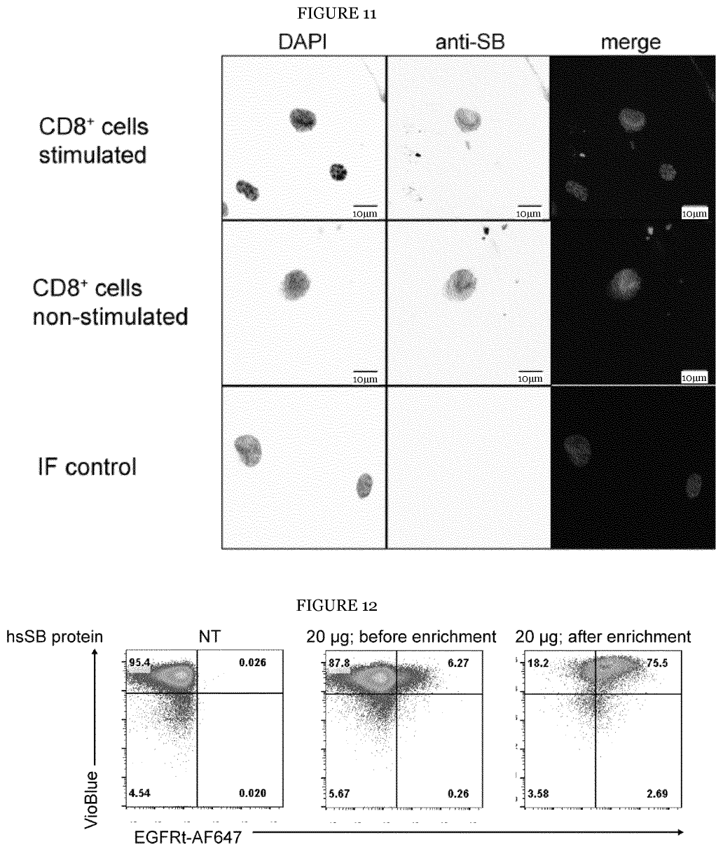

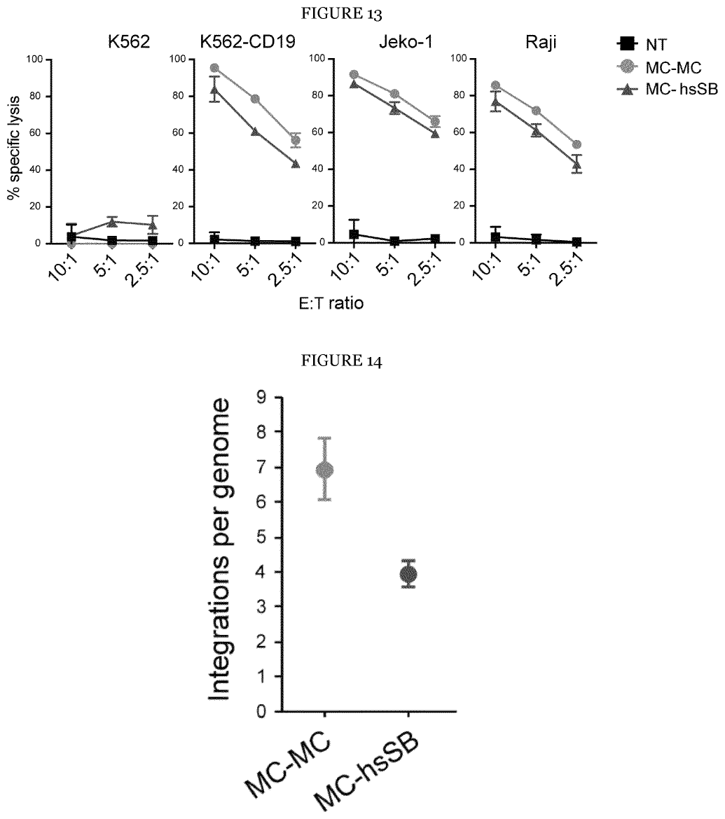

Finally, it was tested whether the intrinsic cell penetration property of hsSB can be exploited for CAR T cell manufacturing (FIG. 10). As electroporation is a stress factor for T cells, hsSB penetration could help preserve their fitness for downstream clinical use. The inventors first analyzed hsSB penetration in primary T cells by immunofluorescence imaging, which showed efficient protein uptake in both stimulated and non-stimulated cells within 3 hours (FIG. 11). hsSB efficiently entered the nucleus also in non-dividing cells, consistent with active transport using its intrinsic nuclear localization signal. To probe transposition, T cells were electroporated with CD19 CAR MC and hsSB was added to the cell culture media. This successfully generated human CD8+ CD19 CAR T cells at an overall transgenesis frequency of 5-7% (FIG. 12). CAR T cells were then enriched up to 90% purity by MACS (44) and showed potent lysis of CD19+ target cells, as well as high levels of effector cytokine secretion (FIGS. 12, and 13). Cells produced with this procedure showed an average number of four insertions, which is lower compared to the CAR MC-SB MC DNA based protocol (6-8 insertions; FIG. 14).

### Example 4: Using the Self-Penetrating Transposase Protein as a Cargo Shuttle into Cells

HeLa cells were seeded onto a Nunc™ Lab-Tek™ II 8-well Chamber Slides™ (Thermo Fisher) (2×104 cells per well in 500 μL DMEM supplemented with 10% (v/v) human serum and 2 mM L-glutamine). On the next day, cells were incubated with hsSB-GFP at a concentration of 0.5 μM in a volume of 250 μL/well serum-free DMEM for 1 hour. Then, media was removed and cells were fixed with PFA 4% in PBS and incubated 30 min with DAPI to visualize the nuclei. Cells were imaged with a Zeiss LSM 780 confocal microscope (using a 63× oil submersion objective) in the ALMF core facility at EMBL Heidelberg. For imaging, the middle part of the nucleus was placed in focus to detect nuclear localization of hsSB.

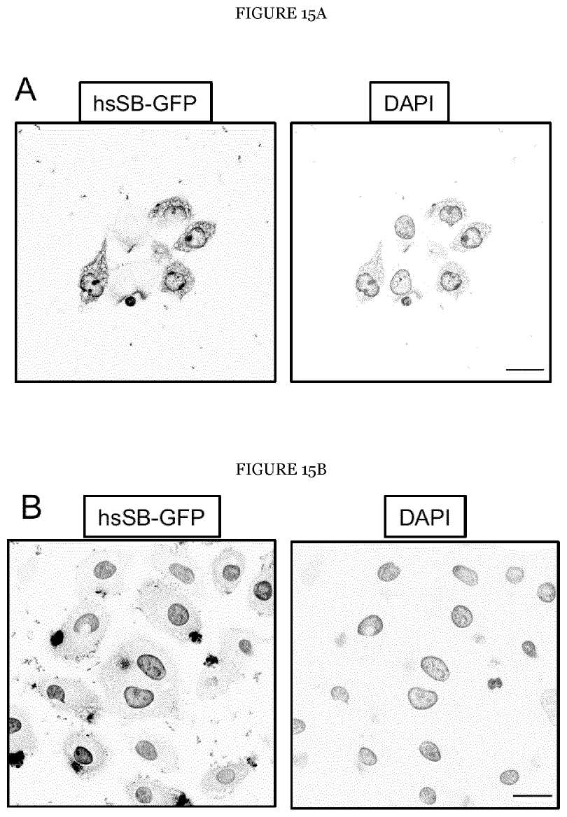

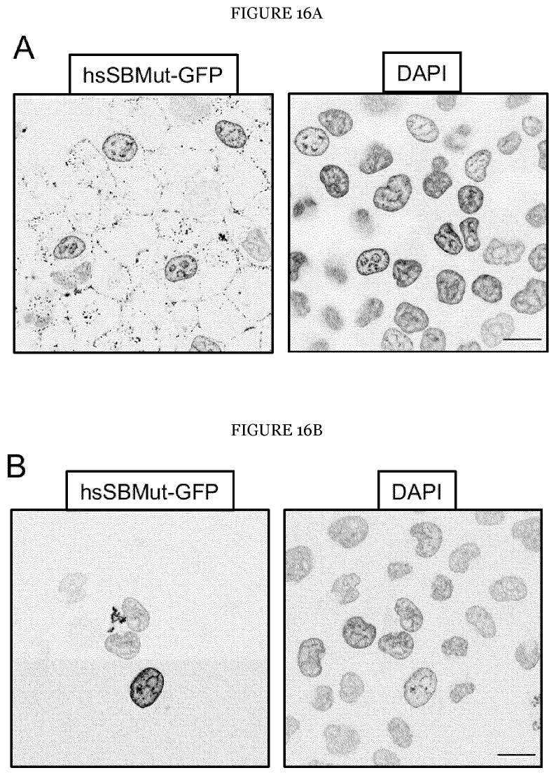

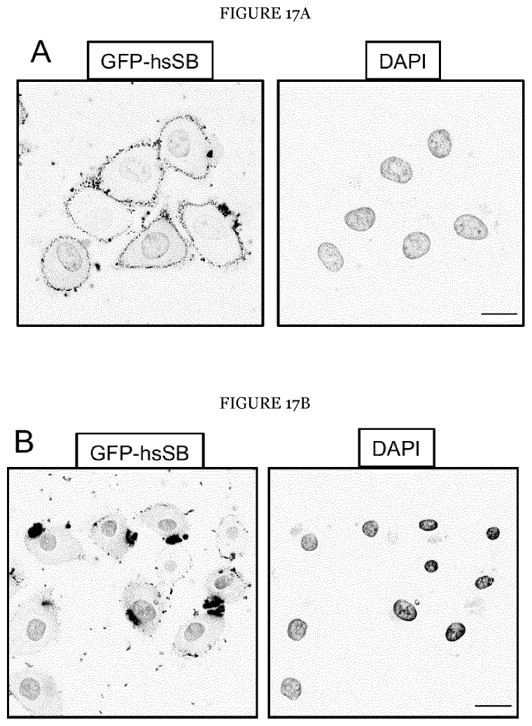

FIG. 15 shows that the hsSB-GFP fusion protein (hsSB fused to the N-terminus of GFP) enters the cells' nuclei within 1 h (A) and is retained at least for the following 24 h (B) as observed by GFP fluorescence imaging. FIGS. 16 A and B show the same effect for a catalytically inactive mutant version of hsSB in HeLa cells. Further, fusing hsSB to the C-terminus of the GFP equally promotes penetration into HeLa cells (FIG. 17).

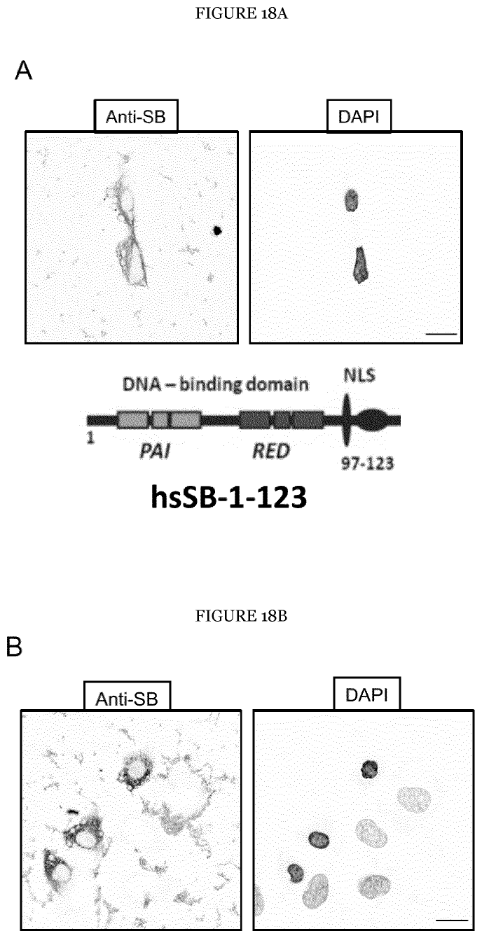

In another experiment a truncated version of the hsSB, namely a version consisting of the DNA binding domain of the protein (bottom of FIG. 18A) is probed in HeLa cells. Results show that the hsSB's DNA binding domain is sufficient for autonomous cell penetration from the culture media. hsSB DBD is detected in the cells with immunofluorescence imaging using an SB-specific antibody. The protein (peptide) enters the cells within 3 h (FIG. 18A) and is retained at least for the following 24 h (FIG. 18B).

### REFERENCES

The references are:
1. C. H. Lamers et al., Immune responses to transgene and retroviral vector in patients treated with ex vivo-engineered T cells. Blood 117, 72-82 (2011).
2. F. Bushman et al., Genome-wide analysis of retroviral DNA integration. Nat Rev Microbiol 3, 848-858 (2005).
3. M. Kosicki, K. Tomberg, A. Bradley, Repair of double-strand breaks induced by CRISPR-Cas9 leads to large deletions and complex rearrangements. Nat Biotechnol 36, 765-771 (2018).
4. E. Haapaniemi, S. Botla, J. Persson, B. Schmierer, J. Taipale, CRISPR-Cas9 genome editing induces a p53-mediated DNA damage response. Nat Med 24, 927-930 (2018).
5. R. J. Ihry et al., p53 inhibits CRISPR-Cas9 engineering in human pluripotent stem cells. Nat Med 24, 939-946 (2018).
6. R. Monjezi et al., Enhanced CAR T-cell engineering using non-viral Sleeping Beauty transposition from minicircle vectors. Leukemia 31, 186-194 (2017).
7. M. Hudecek et al., Going non-viral: the Sleeping Beauty transposon system breaks on through to the clinical side. Crit Rev Biochem Mol Biol 52, 355-380 (2017).
8. M. M. D'Aloia, I. G. Zizzari, B. Sacchetti, L. Pierelli, M. Alimandi, CAR-T cells: the long and winding road to solid tumors. Cell Death Dis 9, 282 (2018).
9. Z. Ivics, P. B. Hackett, R. H. Plasterk, Z. Izsvak, Molecular reconstruction of Sleeping Beauty, a Tc1-like transposon from fish, and its transposition in human cells. Cell 91, 501-510 (1997).
10. A. J. Dupuy, N. A. Jenkins, N. G. Copeland, Sleeping beauty: a novel cancer gene discovery tool. Human molecular genetics 15 Spec No 1, R75-79 (2006).
11. R. Hodge, S. A. Narayanavari, Z. Izsvak, Z. Ivics, Wide Awake and Ready to Move: 20 Years of Non-Viral Therapeutic Genome Engineering with the Sleeping Beauty Transposon System. Human gene therapy 28, 842-855 (2017).
12. K. Kawakami, D. A. Largaespada, Z. Ivics, Transposons As Tools for Functional Genomics in Vertebrate Models. Trends in genetics: TIG 33, 784-801 (2017).
13. P. Kebriaei, Z. Izsvak, S. A. Narayanavari, H. Singh, Z. Ivics, Gene Therapy with the Sleeping Beauty Transposon System. Trends in genetics: TIG 33, 852-870 (2017).
14. C. J. Turtle et al., Durable Molecular Remissions in Chronic Lymphocytic Leukemia Treated With CD19-Specific Chimeric Antigen Receptor-Modified T Cells After Failure of Ibrutinib. J Clin Oncol 35, 3010-3020 (2017).
15. C. A. Klebanoff, S. A. Rosenberg, N. P. Restifo, Prospects for gene-engineered T cell immunotherapy for solid cancers. Nat Med 22, 26-36 (2016).
16. J. Tipanee, Y. C. Chai, T. VandenDriessche, M. K. Chuah, Preclinical and clinical advances in transposon-based gene therapy. Biosci Rep, (2017).
17. P. Kebriaei et al., Phase I trials using Sleeping Beauty to generate CD19-specific CAR T cells. The Journal of clinical investigation 126, 3363-3376 (2016).
18. M. Galla et al., Avoiding cytotoxicity of transposases by dose-controlled mRNA delivery. Nucleic acids research 39, 7147-7160 (2011).
19. X. Huang et al., Unexpectedly high copy number of random integration but low 50 frequency of persistent expression of the Sleeping Beauty transposase after trans delivery in primary human T cells. Human gene therapy 21, 1577-1590 (2010).
20. Q. Liang, J. Kong, J. Stalker, A. Bradley, Chromosomal mobilization and reintegration of Sleeping Beauty and PiggyBac transposons. Genesis 47, 404-408 (2009).
21. A. Wilber et al., Messenger RNA as a source of transposase for sleeping beauty transposon-mediated correction of hereditary tyrosinemia type I. Molecular therapy the journal of the American Society of Gene Therapy 15, 1280-1287 (2007).
22. Z. Jin et al., The hyperactive Sleeping Beauty transposase SB100X improves the genetic modification of T cells to express a chimeric antigen receptor. Gene therapy 18, 849-856 (2011).
23. M. Holstein et al., Efficient Non-viral Gene Delivery into Human Hematopoietic Stem Cells by Minicircle Sleeping Beauty Transposon Vectors. Molecular therapy: the journal of the American Society of Gene Therapy 26, 1137-1153 (2018).
24. H. Zayed, Z. Izsvak, D. Khare, U. Heinemann, Z. Ivics, The DNA-bending protein HMGB1 is a cellular cofactor of Sleeping Beauty transposition. Nucleic acids research 31, 2313-2322 (2003).
25. A. Hendel et al., Chemically modified guide RNAs enhance CRISPR-Cas genome editing in human primary cells. Nat Biotechnol 33, 985-989 (2015).
26. T. Gaj, J. Guo, Y. Kato, S. J. Sirk, C. F. Barbas, 3rd, Targeted gene knockout by direct delivery of zinc-finger nuclease proteins. Nature methods 9, 805-807 (2012).
27. M. Trubitsyna et al., Use of mariner transposases for one-step delivery and integration of DNA in prokaryotes and eukaryotes by transfection. Nucleic acids research 45, e89 (2017).
28. A. O. Paatero et al., Bacteriophage Mu integration in yeast and mammalian genomes. Nucleic acids research 36, e148 (2008).
29. T. S. Rasila et al., Mu transpososome activity-profiling yields hyperactive MuA variants for highly efficient genetic and genome engineering. Nucleic acids research 46, 4649-4661 (2018).
30. Y. Cai et al., DNA transposition by protein transduction of the piggyBac transposase from lentiviral Gag precursors. Nucleic acids research 42, e28 (2014).
31. K. A. Skipper et al., Time-Restricted PiggyBac DNA Transposition by Transposase Protein Delivery Using Lentivirus-Derived Nanoparticles. Molecular Therapy—Nucleic Acids 11, 253-262 (2018).
32. S. Kim, D. Kim, S. W. Cho, J. Kim, J. S. Kim, Highly efficient RNA-guided genome editing in human cells via delivery of purified Cas9 ribonucleoproteins. Genome Res 24, 1012-1019 (2014).
33. J. A. Zuris et al., Cationic lipid-mediated delivery of proteins enables efficient protein-based genome editing in vitro and in vivo. Nat Biotechnol 33, 73-80 (2015).
34. L. Mates et al., Molecular evolution of a novel hyperactive Sleeping Beauty transposase enables robust stable gene transfer in vertebrates. Nature genetics 41, 753-761 (2009).
35. F. Voigt et al., Sleeping Beauty transposase structure allows rational design of hyperactive variants for genetic engineering. Nat Commun 7, 11126 (2016).
36. V. Avramopoulou, A. Mamalaki, S. J. Tzartos, Soluble, oligomeric, and ligand-binding extracellular domain of the human alpha7 acetylcholine receptor expressed in yeast: replacement of the hydrophobic cysteine loop by the hydrophilic loop of the ACh-binding protein enhances protein solubility. The Journal of biological chemistry 279, 38287-38293 (2004).
37. M. Cozzolino et al., Cysteine 111 affects aggregation and cytotoxicity of mutant Cu,Zn-superoxide dismutase associated with familial amyotrophic lateral sclerosis. The Journal of biological chemistry 283, 866-874 (2008).
38. H. Slusarczyk, S. Felber, M. R. Kula, M. Pohl, Stabilization of NAD-dependent formate 50 dehydrogenase from Candida boidinii by site-directed mutagenesis of cysteine residues. Eur J Biochem 267, 1280-1289 (2000).
39. X. Xie et al., Rational improvement of simvastatin synthase solubility in Escherichia coli leads to higher whole-cell biocatalytic activity. Biotechnol Bioeng 102, 20-28 (2009).
40. Z. Izsvak, Z. Ivics, R. H. Plasterk, Sleeping Beauty, a wide host-range transposon vector 55 for genetic transformation in vertebrates. Journal of molecular biology 302, 93-102 (2000).
41. M. L. Davila et al., Efficacy and toxicity management of 19-28z CAR T cell therapy in B cell acute lymphoblastic leukemia. Sci Transl Med 6, 224ra225 (2014).
42. S. L. Maude et al., Chimeric antigen receptor T cells for sustained remissions in leukemia. N Engl J Med 371, 1507-1517 (2014).
43. D. W. Lee et al., T cells expressing CD19 chimeric antigen receptors for acute lymphoblastic leukaemia in children and young adults: a phase 1 dose-escalation trial. Lancet 385, 517-528 (2015).
44. X. Wang et al., A transgene-encoded cell surface polypeptide for selection, in vivo tracking, and ablation of engineered cells. Blood 118, 1255-1263 (2011).
45. Z. Ivics, Z. Izsvak, A. Minter, P. B. Hackett, Identification of functional domains and evolution of Tc1-like transposable elements. Proceedings of the National Academy of Sciences of the United States of America 93, 5008-5013 (1996).
46. S. Charrier et al., Quantification of lentiviral vector copy numbers in individual hematopoietic colony-forming cells shows vector dose-dependent effects on the frequency and level of transduction. Gene therapy 18, 479-487 (2011).
47. G. Luo, Z. Ivics, Z. Izsvak, A. Bradley, Chromosomal transposition of a Tc1/mariner-like element in mouse embryonic stem cells. Proceedings of the National Academy of Sciences of the United States of America 95, 10769-10773 (1998).
48. P. B. Hackett, D. A. Largaespada, K. C. Switzer, L. J. Cooper, Evaluating risks of insertional mutagenesis by DNA transposons in gene therapy. Transl Res 161, 265-283 (2013).
49. J. Chusainow et al., A study of monoclonal antibody-producing CHO cell lines: what makes a stable high producer? Biotechnol Bioeng 102, 1182-1196 (2009).
50. T. L. Lu et al., A Rapid Cell Expansion Process for Production of Engineered Autologous CAR-T Cell Therapies. Hum Gene Ther Methods 27, 209-218 (2016).
51. L. Gattinoni et al., A human memory T cell subset with stem cell-like properties. Nat Med 17, 1290-1297 (2011).
52. C. A. Klebanoff, L. Gattinoni, N. P. Restifo, Sorting through subsets: which T-cell populations mediate highly effective adoptive immunotherapy? J Immunother 35, 651-660 (2012).
53. C. J. Turtle et al., CD19 CAR-T cells of defined CD4+:CD8+ composition in adult B cell ALL patients. The Journal of clinical investigation 126, 2123-2138 (2016).
54. M. V. Maus et al., T cells expressing chimeric antigen receptors can cause anaphylaxis in humans. Cancer Immunol Res 1, 26-31 (2013).
55. H. Singh, H. Huls, P. Kebriaei, L. J. Cooper, A new approach to gene therapy using Sleeping Beauty to genetically modify clinical-grade T cells to target CD19. Immunol Rev 257, 181-190 (2014).
56. O. Makarova, E. Kamberov, B. Margolis, Generation of deletion and point mutations with one primer in a single cloning step. BioTechniques 29, 970-972 (2000).
57. S. Ruf et al., Large-scale analysis of the regulatory architecture of the mouse genome with a transposon-associated sensor. Nature genetics 43, 379-386 (2011).
58. M. Hudecek et al., The nonsignaling extracellular spacer domain of chimeric antigen receptors is decisive for in vivo antitumor activity. Cancer Immunol Res 3, 125-135 (2015).
59. M. Hudecek et al., The B-cell tumor-associated antigen ROR1 can be targeted with T cells modified to express a ROR1-specific chimeric antigen receptor. Blood 116, 4532-4541 (2010).
60. M. Hudecek et al., Receptor affinity and extracellular domain modifications affect tumor recognition by ROR1-specific chimeric antigen receptor T cells. Clin Cancer Res 19, 3153-3164 (2013).
61. H. Li, R. Durbin, Fast and accurate short read alignment with Burrows-Wheeler transform. Bioinformatics 25, 1754-1760 (2009).
62. A. R. Quinlan, I. M. Hall, BEDTools: a flexible suite of utilities for comparing genomic features. Bioinformatics 26, 841-842 (2010).
63. J. T. Robinson et al., Integrative genomics viewer. Nat Biotechnol 29, 24-26 (2011).
64. W. Huber et al., Orchestrating high-throughput genomic analysis with Bioconductor. Nature methods 12, 115-121 (2015).
65. B. Langmead, C. Trapnell, M. Pop, S. L. Salzberg, Ultrafast and memory-efficient alignment of short DNA sequences to the human genome. Genome biology 10, R25 (2009).
66. C. W. Chiang et al., Ultraconserved elements in the human genome: association and transmission analyses of highly constrained single-nucleotide polymorphisms. Genetics 192, 253-266 (2012).

1. A method for genetically engineering a target biological cell, the method comprising in any sequence the steps of: (i) introducing a transposon construct into the biological cell and/or providing a biological cell comprising a transposon construct; and (ii) contacting the target biological cell with a transposase protein in absence of, or without using, a protein transfection procedure or protein transfection reagent.

2. The method according to claim 1 or 2, wherein the transposase protein is, or is derived from, a Sleeping Beauty (SB) transposase.

3. The method according to claim 2, wherein the SB transposase is a protein comprising a sequence having at least 80% sequence identity to a sequence shown in any of SEQ ID NO: 1 to 3.

4. The method according to any one of claims 1 to 3, wherein the transposase protein consists of, or consists essentially of, and amino acid sequence shown in any one of SEQ ID NO: 1 to 3, optionally with not more than 50 amino acid substitutions, additions, insertions, deletions or inversions, compared to these sequences.

5. The method according to any one of claims 1 to 4, wherein the transposase protein is provided by adding the transposase protein to a medium in which said biological cell is contained, preferably to a cell culture medium of the target biological cell.

6. The method according to any one of claims 1 to 5, wherein the target biological cell is a mammalian cell, preferably selected from a stem cell, such as a hematopoietic stem cell, embryonic stem cell, spontaneously immortalized cell, artificial immortalized cell, primary cell (neurons, resting T cells), a cell derived from a B-cell such as plasma cells, Chinese hamster ovary (CHO) cell, induced pluripotent stem cell (iPSC), or is an immune cell, such as a T lymphocyte, preferably a CD4 or CD8 positive T cell, or is a Natural Killer (NK) cell, a macrophage, a dendritic cell or a B-cell.

7. The method according to any one of claims 1 to 6, wherein the transposon comprises a protein encoding nucleotide sequence, such as a sequence encoding for an antibody, a T cell receptor, or a chimeric antigen receptor (CAR).

8. A method for the delivery of a cargo-compound into a biological cell, the method comprising, covalently or non-covalently and directly or indirectly, attaching a cargo-compound to a shuttle protein to obtain a cargo-shuttle complex, and contacting the biological cell with the cargo-shuttle complex; characterized in that the shuttle protein comprises a transposase protein sequence, preferably a transposase protein as defined in any of claims 1 to 7.

9. The method according to any claim 8, wherein the shuttle protein is covalently or non-covalently coupled to a linker compound, preferably wherein the linker compound is suitable for covalently or non-covalently coupling the cargo-compound to the shuttle protein.

10. The method according to claim 9, wherein the linker compound is selected from a sortase donor or acceptor site, a biotin or streptavidin protein, or a functionally alternative component of a protein coupling system.

11. The method according to any one of claims 8 to 10, wherein the cargo-compound is selected from a small molecule, a macro-molecule, a peptide, a polypeptide, a protein, a nucleic acid, such as an RNA, DNA, RNA-DNA hybrid, PNA, or is a sugar compound, a fatty acid containing compound.

12. A use of a transposase protein in the delivery of a cargo-compound into a biological cell, wherein the transposase protein is used as a cellular shuttle protein and is covalently or non-covalently and directly or indirectly attached to the cargo-compound.

13. A cellular-shuttle, comprising
(i) a transposase protein covalently or non-covalently coupled to a cargo compound; or
(ii) a transposase protein covalently or non-covalently coupled to a linker compound, and wherein the linker compound is suitable for the covalent or non-covalent coupling of the cellular-shuttle to a cargo compound; or
(iii) a transposase protein covalently or non-covalently coupled to a linker compound, and wherein the linker compound is further covalently or non-covalently coupled to a cargo-compound.

14. A method for introducing a transposase protein into a biological cell, the method comprising contacting the cell with the transposase protein in absence of a protein transfection agent or without using a protein transfection procedure, such as electroporation.

15. The method according to claim 14, wherein the transposase protein is a transposase protein as defined in any one of the preceding claims.
